(19) United States
(12) Patent Application Publication
(10) Pub. No.: US 2022/0051767 A1
(43) Pub. Date: Feb. 17, 2022

(54) PERSONALIZED INSTRUMENTATION MANUFACTURE LEDGER TRACKING

(71) Applicant: Zimmer, Inc., Warsaw, IN (US)

(72) Inventor: Vinay Tikka, Winona Lake, IN (US)

(21) Appl. No.: 17/401,796

(22) Filed: Aug. 13, 2021

Related U.S. Application Data
(60) Provisional application No. 63/065,830, filed on Aug. 14, 2020.

(51) Int. Cl. G16H 10/60; H04L 9/32

(52) U.S. Cl. CPC G16H 10/60; H04L 2209/38; H04L 9/3247; H04L 9/3236

(57) ABSTRACT

A system or method may be used to track a patient throughout a treatment journey. A ledger may be used to store data related to aspects of the treatment journey. The ledger may include a plurality of blocks, with each block corresponding to a particular treatment event. A new block may be added to the ledger when a treatment event occurs. The new block may include patient information from the treatment event. The new block may be signed, for example in response to receiving verification from an entity that is an authority on the patient information. The ledger may be stored with the new block.

### CLAIM OF PRIORITY

This application claims the benefit of priority to U.S. Provisional Application No. 63/065,830 filed Aug. 14, 2020, titled “PERSONALIZED INSTRUMENTATION MANUFACTURE LEDGER TRACKING,” which is hereby incorporated herein by reference in its entirety.

### BACKGROUND

Patient specific guides or implants may be used during orthopedic surgery to improve patient outcomes. A surgeon may use patient specific tools or techniques based on planning, 3D printing, and imaging to conduct a personalized experience for a patient. For example, patient imaging may be used to generate a 3D virtual model for an anatomic feature. Virtual planning may use a 3D model or a 3D printed guide. Patient Specific Instruments are used to accurately and reproducibly guide fixation. These instruments may be generated based on medical imaging of the patient with mechanical axis-based pin guides conforming precisely to the patient's anatomy.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a diagram showing an overview of personalized implant creation and supporting infrastructure for tracking treatment data in accordance with some embodiments.

FIG. 2 illustrates a ledger including example block entries for tracking treatment data in accordance with some embodiments.

FIG. 3 illustrates a chain of a distributed ledger in progress in accordance with some embodiments.

FIG. 4 illustrates a chain of a distributed ledger including a branching path in accordance with some embodiments.

FIG. 5 illustrates a flowchart illustrating a technique for tracking a patient throughout a treatment journey in accordance with some embodiments.

FIG. 6 illustrates a block diagram of an example machine upon which any one or more of the techniques discussed herein may perform in accordance with some embodiments.

### DETAILED DESCRIPTION

Systems and methods for tracking treatment data throughout a treatment journey for an orthopedic patient are described herein. The systems and methods herein may include using a blockchain or ledger to track or store data related to the treatment journey. A blockchain is a list of records including blocks, that are compiled in a chain, also known as a distributed ledger. A ledger includes entries, and may be stored in a distributed manner or in a local storage. A table may be stored in a database to keep track of entries (e.g., blocks) within a blockchain or other type of ledger. A difference between a distributed ledger and a local ledger is that the distributed ledger includes entries stored in more than one location, such as distributed among different entities, servers, or the like. A distributed ledger may use cryptography to securely store entries such that the entries may not be accessed by unauthorized users. In an example, a distributed ledger or blockchain may use a voting mechanism among entities storing the distributed ledger or blockchain to verify accuracy of blocks or entries.

A treatment journey for an orthopedic patient may include a number of steps, which may be discrete or overlapping. For example, a typical patient may begin to experience pain, schedule an initial visit with a doctor, be referred, have imaging taken (e.g., an x-ray), meet with a surgeon, schedule a procedure (e.g., to insert an implant), have post-operative care (e.g., physical therapy), or the like. The treatment journey may last years, for example when an additional procedure occurs (e.g., a revision on a joint) or when physical therapy continues. The treatment journey may include interactions with different entities, each of which has different competencies. In order to track the treatment journey and maintain an accurate historical record, a ledger for the patient may be created and used, with for example, a block created and signed by each entity for each step along the treatment journey. This trail may be audited to ensure compliance, record-keeping standards, or to review a patient outcome.

In some cases, a centralized server holds information related to a case, and files related to the case may be stored in a separate location. Decentralized storing of the process instead of single point allows for better transparency and a tamper-proof audit trail, which may be used for regulatory audits.

In an example, a decentralized ledger may be used for activities related to personalized implant, trial/provisional implant, bone models, or another manufactured device (e.g., a patient-specific instrument). Using the decentralized nature of the ledger, as well as inherent security built into a distributed ledger, activities committed to the ledger are tamper-proof. A ledger may be used to store assets, such as DICOM files, 3D model files, patient plans, data (e.g., operation results, patient feedback, compliance with post-operative treatment, etc.) or the like.

A ledger may be used to give access to different users, such as surgeons to write or read a file at an appropriate step, for example at plan approval, patient identification for manufacturing, scan techs for image upload, or the like. Each user machine may be used as a node to create a block, store content in the block, or sign the block. After a procedure is performed, the implant identifier or instrument identifier may be added to the ledger (e.g., in a block corresponding to execution of the procedure), along with other procedure-related information.

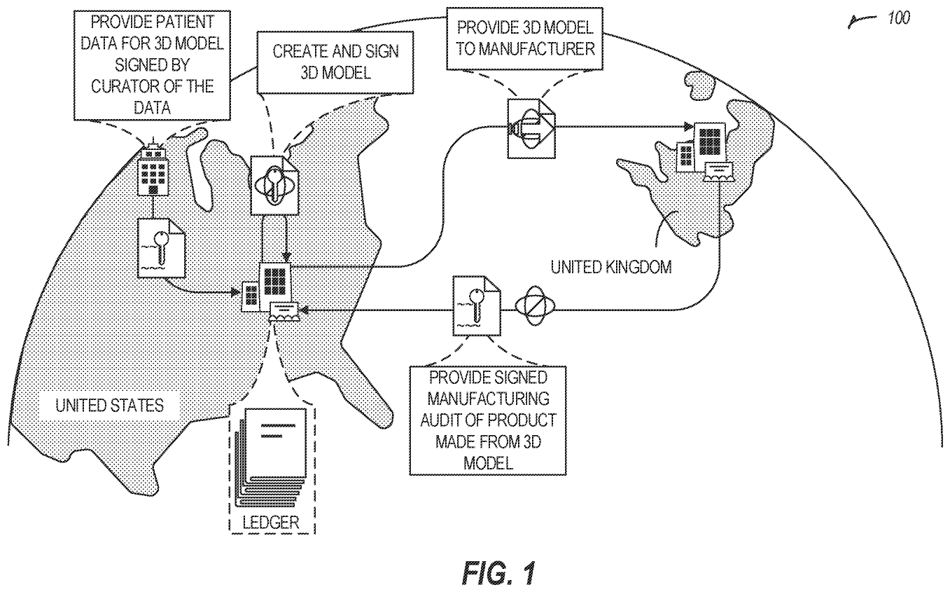

FIG. 1 illustrates a diagram 100 showing an overview of personalized implant creation and supporting infrastructure for tracking treatment data in accordance with some embodiments. The diagram 100 illustrates a global example of the distributed nature of a ledger. While the diagram 100 shows the example of activities in two different countries, in other examples all activities may be conducted within a single country, or even a single building. Patient treatment data may be generated, stored, and signed to blocks. For example, patient data (e.g., imaging) may be provided and signed in a first block, 3D model generation information may be stored at a second block, manufacturing information (e.g., of a trial or implant based on the 3D model) may be stored at a third block, etc. Each block may be signed by the respective entity that generated the data and created the block. The ledger may be stored (e.g., after a currently last block may be signed, though additional blocks may subsequently added) in each of the locations, with communication among the different entities. In other examples, the ledger may be stored at each location only up to the point where the entity associated with that location has completed its block (e.g., subsequent blocks may not necessarily be sent back to devices associated with previous steps). A complete copy of the ledger may be stored at an auditable location.

The steps shown in FIG. 1 are generalized, and may include more or fewer steps. In an example, patient-related information in the form of case details, medical images like X-ray, CT, MRI may be generated and stored in blocks. This information may be processed to create a patient-specific instrument or a surgical plan, either or both of which may be stored in a block. Further steps may include image acquisition, segmentation, plan creation, plan approval, or guide creation. These steps may be included in separate blocks or some may be combined.

The ledger may include a patient- or surgery-specific chain. For example, a patient may have a chain including multiple surgical procedures (e.g., a left knee and a right knee arthroplasty). In other examples, a chain may be limited to a single procedure (e.g., the patient may have a first chain for a left knee and a second chain for a right knee arthroplasty). In other examples, the chain may include multiple branches (e.g., a base block for patient information, but two separate chains for a left knee and a right knee arthroplasty). The ledger may include a one-way chain that moves from supplier to seller to surgeon to patient (and optionally back to a surgeon) as a way of tracking patient data. The one-way nature of the chain does not refer to movement of the ledger, but instead to security of the ledger. For example, once a block is signed, it may not be modified (except optionally in some examples by the signer of the block). Each link in the chain may provide a transaction certifying that a part (e.g., an action taken by an entity) is complete and accurate (e.g., a manufacturer certifies an implant, imaging company certifies the patient image, etc.).

A patient-specific ledger may include images, data, files, etc., for tracking over time. In an example, specific aspects of the ledger (e.g., a block) may be shared with an entity (e.g., a surgeon). The sharing may be done in a secured manner, including cryptographically storing and sending information. The block may be sent to a surgeon for a consultation (e.g., an expert opinion, second opinion, etc.), or as a referral. For a referral, a patient with an issue may seek out a surgeon using the block, rather than the typical referral process from a primary doctor. The patient may seek information from surgeons on cost, time, expertise, or the like, for a surgery. This type of referral process may be undertaken by the patient, and may take patient preferences into account. A surgeon may return a bid or information related to how the surgical procedure would be performed by that surgeon. A second block may be created by the surgeon returning the bid, or the information may be sent in another manner. When each step of the referral or surgical process is completed, the block may be closed. The patient may then seek out a next bid for a next step. A bid may be non-binding (e.g., an estimate or statement of intent) or binding (e.g., an offer). The ledger may be used to distribute the bidding process to remove the potential inaccuracies or overhead of first consulting a primary doctor to find all referrals. The ledger also allows for the patient to make more decisions at each step rather than be locked into a single path based on a primary doctor decision or a surgeon decision.

Other aspects of a surgical procedure may be tracked using a ledger. For example, an instrument, such as a trial, a placement device, or an implant may be tracked in a chain throughout the conceptual, manufacturing, or surgical process. In an example, a personalized instrument may be used in a surgical procedure. The personalized instrument may be tracked in a ledger, with a block for each step of the life of the instrument. For example, a surgeon may create a block with patient-specific data, indicating a personalized instrument is needed. A next block may be a 3D rendering of the personalized instrument (which may be certified as corresponding to the patient-specific data). A next block may include a certification from a manufacturer that a physical personalized instrument corresponds to the 3D rendering. An administrative block or blocks may be used to track the travel of the personalized instrument from manufacturing to surgical field, ready for use. A surgeon may use the personalized instrument, and create a block indicating the use (including, optionally feedback about the personalized instrument). Disposal or recycling of the personalized instrument may be indicated in another block.

The blocks in the ledger may be hashed. Each block may have its own encryption scheme, or all blocks in a particular ledger may use the same encryption scheme. For example, each block may be encrypted with a public key, only decryptable with a private key for that block. Various cryptographic techniques may be used, such that, for example only an entity that signed and created or edited a block may decrypt the block. In some examples, a master key may be available (or a set of private keys) such that a central entity may decrypt all blocks, while entities creating and storing blocks may only decrypt blocks they create. This allows for the ledger to be audited, while preventing data from being released to entities that did not create the data. In an example, keys, block information, or ledger information may be stored at a central location, though the ledger itself is distributed. For example, a database may store key-hash combinations and identify which blocks each key-hash corresponds to, and the database may be accessible only to a particular entity.

In an example, a block may include a smart contract. A smart contract is a blockchain-based contract system, that executes when a set of conditions are met. For example, in the context of an instrument or patient ledger, a smart contract may include manufacture of a personalized instrument on one side, and payment for the personalized instrument on the other side of the contract. When the personalized instrument is indicated as delivered, the smart contract may release the payment. Other uses of the smart contract concept in this ledger environment may include a patient signing up to have a ledger, which may include indicating what information is to be protected (e.g., personal health information), what entities may access which blocks, audit provisions, where data is stored, etc. In an example, a blockchain-based contract system may use an existing platform (e.g., for a smart contract), such as Ethereum or Bitcoin.

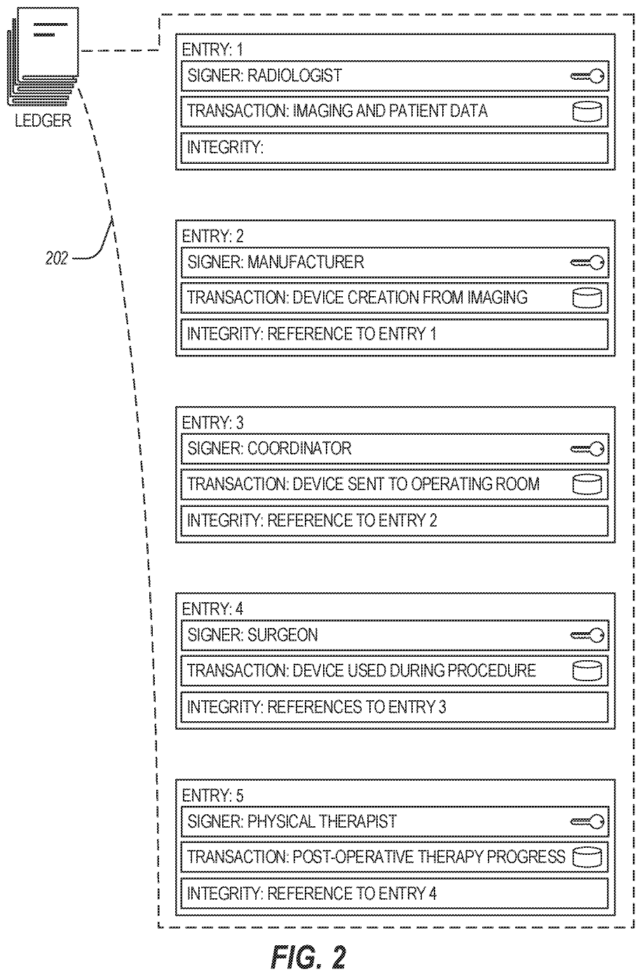

FIG. 2 illustrates a ledger 202 including example block entries for tracking treatment data in accordance with some embodiments. The ledger 202 includes a plurality of blocks, which may be used to store data related to a patient. Each block in the ledger 202 may correspond to a transaction (a data creation event), an entity (e.g., a manufacturer, a doctor, etc.), or a device (e.g., a trial, an implant, an instrument, etc.).

The ledger 202 includes example blocks, such as entry 1, which includes an imaging transaction signed by a radiologist. Entry 1 may be generated when a medical image of a patient (e.g., an x-ray, CT, MR, etc.) is generated or read by a radiologist. The radiologist may include notes about the image. In another example, entry 1 may be signed by a technician responsible for capturing the image (e.g., the image does not necessarily need to be read by a radiologist for this block). Entry 1 may include patient data (e.g., demographic information, assessment data such as pain, or personal information). In an example, entry 1 is the first entry in the ledger 202, which represents first captured data related to a patient treatment journey. In other examples, the first entry may include only patient data such as personal information. In still other examples, the treatment journey may begin with a different block, such as a doctor referral, a medical history, a patient chart (e.g., the block may be populated by previously captured data), or the like. When entry 1 is completed, that block may be signed, for example by a technician or a radiologist. The signer may certify that the data is complete or correct. In another example, the signer may merely close the block (e.g., encrypt the block) rather than certify that any information is complete or correct. In yet another example, the signer may encrypt the block and certify the data in the block. Different options for signing a block may be used together in the ledger 202.

Entry 2 in the ledger 202 may represent a next step in the patient treatment journey, for example at a later time or date. In another example, entry 2 may overlap in time or date with entry 1 (e.g., both may be open at the same time). In the example shown in the ledger 202, entry 1 is signed before entry 2 is signed. Entry 2 shows a manufacturer of a device (e.g., of a trial, an implant, an instrument, or the like), which may be personalized to the patient. The manufacturer may use the imaging information to manufacture the device. Once the device is manufactured, the manufacturer may sign entry 2. The manufacturer signing a block may indicate that the image data from entry 1 was used to create the device (e.g., a personalized trial or implant). In another example, the manufacturer signing the block may indicate that a device has been generated (e.g., without certifying that the device was generated from the imaging). Entry 2 may include a serial number of the manufactured device, an image of the device, or the like. Entry 2 includes an integrity report, which refers to entry 1. The integrity report signifies within the ledger 202 that entry 2 comes after, depends from, was created after, or refers to entry 1. In the ledger 202, a series of blocks are created or signed in a particular order, for example with entry 2 created or signed after entry 1. The entries may be ordered based on when they are created or when they are signed.

Entry 3, entry 4, and entry 5 include details related to different transactions or events throughout a patient treatment journey, such as delivery of a device, use of a device during a surgical procedure, or post-operative care. The entries may be signed by relevant entities, such as a surgeon or physical therapist. Entries 3-5 may include integrity details similar to entry 2, as described above.

Blocks in the ledger 202 may include details about a single patient for a single treatment journey (e.g., a left knee arthroplasty), a single patient throughout multiple treatments (e.g., a left knee arthroplasty and a right knee arthroplasty), or multiple patients (e.g., for a single surgeon). Blocks may include data, such as details of various events during treatment. The data may include information related to a diagnosis, imaging (e.g., a DICOM image of an x-ray, CT, MR, etc.), a smart contract (e.g., between an imaging company and a manufacturer, between a patient and a surgeon, or the like), personalized instrument flow (e.g., receive and store a DICOM image in a block, generate a personalized instrument model for storing in a block, and save implant metadata, such as a serial number, manufacturer, manufacturing date, or the like in a block). Information that may be added to a block may include patient history, diagnosis, imaging (e.g., DICOM image, such as of an x-ray, CT, MR, etc.), implant design, modeling, implant manufacturing, preoperative plan, intraoperative plan (e.g., adjustments), landmarking, validating cuts, trial insertion, implant insertion, postoperative assessment, physical therapy, outcome assessment, or the like.

During a patient's visit to a hospital or physician office, patient information is collected along with files like X-ray, CT, MRIs to determine the cause of the patient's issue. This information is stored in systems of electronic medical records, over which the patient has no control over who can view or access it. With the increase in privacy laws and data breaches around protected health information, there is no easy way for hospitals to control to prevent and restrict access to these systems once data leaves their systems. The ledger 202 system allows the use of a public key infrastructure and public-key cryptography to encrypt patient data and files, where only authorized users and systems can decrypt the data and access it.

In a typical hospital environment, patient information is stored in an electronic medical records system. The electronic medical records systems may also connect to other systems like labs or accounting in a hospital. In cases of some specialties like orthopedics or oncology, when a clinician decides to do a pre-plan or order a patient-specific device from a medical device company, there is a need to send medical images to the medical devices companies or third party that is outside the hospital infrastructure. Once this information leaves the hospital systems, there is no oversight from the hospital though they are bound by legal contracts between the hospital and medical device manufacturer.

A Public Key Infrastructure (PKI) may be used with the ledger 202 to cryptographically sign a block to keep patient information secure, and only accessible by intended parties. Further, using PKI with the ledger 202 allows audits to be performed to ensure that no data has changed or been breached. PKI may be implemented using an open source certificate authority or certification authority, such as OpenCA from OpenCA PKI Research Labs.

When two parties communicate using public-key encryption, the sender uses the public key belonging to the recipient and uses it to encrypt the information. On receipt of the encrypted data, the recipient uses a private key to decrypt, and thereby access the information. The weak point in this scenario is that the sender has no way to validate that the person who provided them with the public key is who they say they are. The sender has to take entirely on trust the fact that the person they are sending the information to is who they say they are. Using a ledger 202, access may be restricted to particular parties. PKI involves the participation of trusted third parties who verify the identity of the parties wishing to engage in secure communication through the issuing of digital certificates.

For the hospital to control patient-related data, files and data may be encrypted with a Public Key Cryptography. The two main features of the Public Key Cryptography are having a private key and public key. The private key is kept as a secret at the hospital system, and the public key is used by the medical device companies to decrypt the patient data. To control the life cycle of the public key hospital will use the PKI infrastructure. In this scenario, the hospital may be both a CA (Certificate Authority) and RA (Registration Authority). Once a medical device company has contracted with the hospital to share patient data, the medical device company may request a digital certificate from the hospital, which has the public key to decrypt the patient data. The certificate may have an expiration date or time, for example tied to the contract duration. When the hospital wants to revoke access to the patient data, a revoke mechanism from the PKI infrastructure may be used. With the use of the revocation of mechanism in PKI, the hospital may fully control the certificate, which stops medical device companies from having access to the encrypted patient data.

In the above example (which uses a hospital, but a manufacturer or other entity may be substituted for the hospital), entries to the ledger 202 may be signed (e.g., cryptographically signed) by an entity as correct or complete. The ledger 202 itself, or a block or a set of blocks may also be encrypted by a controlling entity to prevent access by the signers after completion of the signing. For example, each entry may be separately signed by a signer, and then encrypted by a controlling entity. The signing may prevent changes to an entry, while the encryption prevents unauthorized access (or any outside access, in some cases). In this example, the signer is prevented from changing the entry by the encryption, while the encrypting entity is prevented from changing the entry by the signing. The encrypting entity may access (but not modify) entries in the ledger 202 by decrypting the entries, for example for auditing the ledger 202.

In another example, the ledger 202 may be distributed (but still optionally stored using encryption, such as by hashing), and the distribution (e.g., using a blockchain with voting) may prevent changes to the ledger 202. The blocks may be stored and voted on in an encrypted format, such that they are not accessible (except to an authorized auditor or other party holding decryption keys). In an example, signing a block may revoke write access to a database, while read access is permitted. An attempt to edit a signed block may not be possible or may result in the voting identifying an unauthorized change. For example, other voters may reject a changed block as not ‘truth’ within the blockchain, and not allow the change to proceed. A timing mechanism may be used to allow changes to a signed block by the signing entity within a particular time frame (e.g., within a few minutes, an hour, a day, a week, etc.), but prevent changes after that time frame.

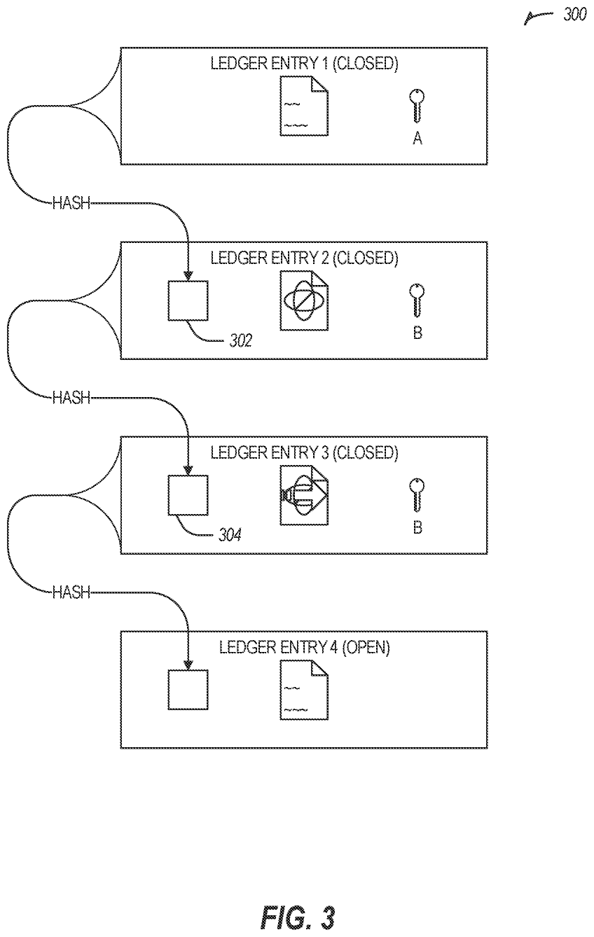

FIG. 3 illustrates a chain of a distributed ledger 300 in progress in accordance with some embodiments. The ledger 300 is in progress, in the sense that ledger entry 4 is open. Open in the ledger 300 may refer to an entry that has been created but not yet signed or encrypted (e.g., not yet hashed). The open entry 4 may include data that has been added or is in progress, but not yet closed and signed. The other entries 1-3 in the ledger 300 are represented as closed, which means they have been signed or encrypted. Entries 2 and 3 are shown as being signed or encrypted with key B, which may correspond to a single entity (e.g., the entity closed both entries), while entry 1 was closed with key A. Entry 4 shows no key, because it is not yet closed.

Each subsequent entry is shown as storing the previous entry (which may be optionally hashed, as shown in FIG. 3). For example, box 302 is a hashed representation of entry 1. Box 304 is a hashed representation of entry 2, which necessarily includes the hashed presentation of entry 1 (because entry 2 includes box 302). Thus, ledger 300 may be stored by saving a version of the last block in the ledger 300 (e.g., a hashed version of the last block).

The ledger 300 may be distributed, such that it is stored in multiple locations or by multiple entities. Each entity or location may vote on the ‘truth’ of the ledger 300, where a majority or a super majority of the voting entities or locations describes the ‘truth’ of the ledger 300. When a change is made (e.g., unauthorized or accidental) by one entity or location, the other entities or locations reject the change by voting against that version of the ledger 300. Thus, unauthorized or accidental changes cannot be made to the ledger 300 (except in extreme cases where multiple entities or locations collude).

A central database may store a table with signing information related to each of the blocks. For example, a hash table or a key table may be stored at the central database to maintain which hashes or keys were used for each block. In an example, an entity that maintains the central database may control access to the ledger 300. For example, an entity that creates or modifies (e.g., saves data to) a block may be prevented from accessing other blocks in the ledger 300. For example, each block may be hashed such that an entity creating a next block can save the hashed previous block but cannot access its contents. Voting by entities may be based on hashed values for each block, rather than contents of the block, allowing for blockchain verification without exposing data. The entity maintaining the central database may have access to all blocks, or at least read access to all blocks, so an audit may be performed. The audit may include querying entities of the blockchain storing the ledger 300 to ensure that the audited copy of the ledger 300 that is accessible to the auditor is verified by the other entities.

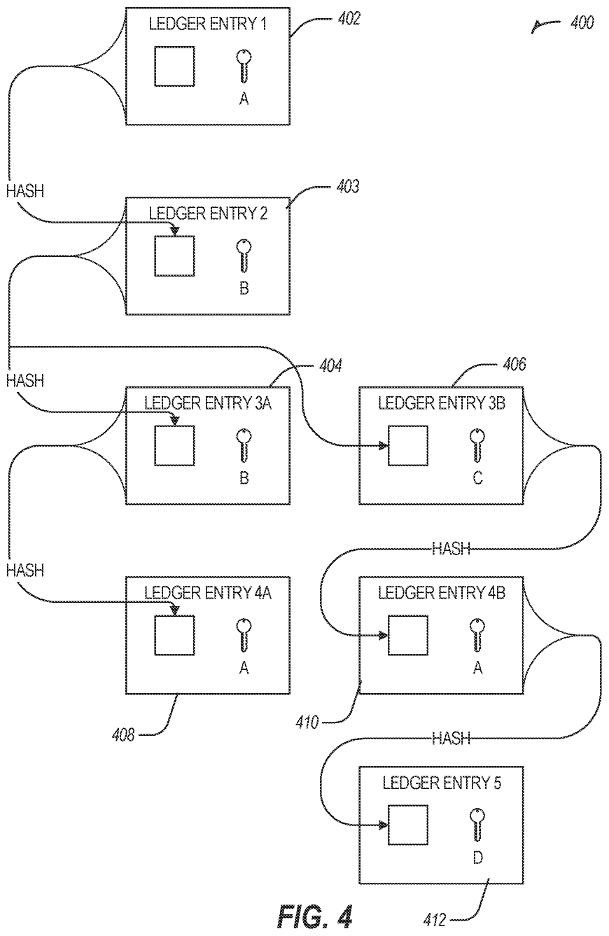

FIG. 4 illustrates a chain of a distributed ledger 400 including a branching path in accordance with some embodiments. The ledger includes a number of blocks, represented as entries 1, 2, 3A, etc. The ledger 400 includes an initial block 402. After block 403, the ledger 400 splits into two different branches, with one branch starting at block 404 and one starting at block 406. The branches may occur when a modification is made (e.g., a change to block 404, such as to rectify a mistake or add more information) or when a new event occurs (e.g., a new treatment journey). Blocks 408 and 410 may have the same content (other than the saved versions of blocks 404 and 406, respectively).

In an example, the ledger 400 stores data through block 406 with entry 4A, with all four blocks 402, 403, 404, and 408 being signed, encrypted, or otherwise closed. At some point, a correction or change is needed for block 404 due to a mistake or additional information at entry 3A. Because one of the attributes of the ledger 400 is that it is an immutable blockchain when a block is closed, the error or addition cannot be made directly to block 404 (since it is closed, and block 408 exists, storing a hashed version of block 404, for example). Instead, a branch is introduced to the ledger 400, creating new block 406 (and block 410 after closing block 406, for example). New block 406 may include data from entry 3A, with modifications or additions as needed. The entire ledger 400, including both branches, may be propagated and saved. The ledger 400 thus includes the historical branch as well as the updated branch (which may be continued on, such as with entry 5 at block 412). The auditable ledger 400 includes both branches for completeness, although the updated branch may be used for further patient treatment and additional entries. The historical branch may be closed to further blocks.

In another example, multiple branches may be used for a single patient during multiple treatment journeys or for multiple patients (e.g., for a single surgeon). In this example, the branches are entirely separate, but share trunk blocks of common data. For example, block 402 may include patient data or preferences, which may not vary between two treatments. Block 403 may include historical imaging that is potentially relevant to both treatments. Then, blocks 404 and 406 may represent respective first entries in each of the two treatments. Further branches may be added for other treatments. In this example, both branches can be added to with additional blocks or entries during treatment. In the multiple patient version of this example, block 402 may include surgeon preferences, with blocks 404 and 406 each being a respective first patient-specific block.

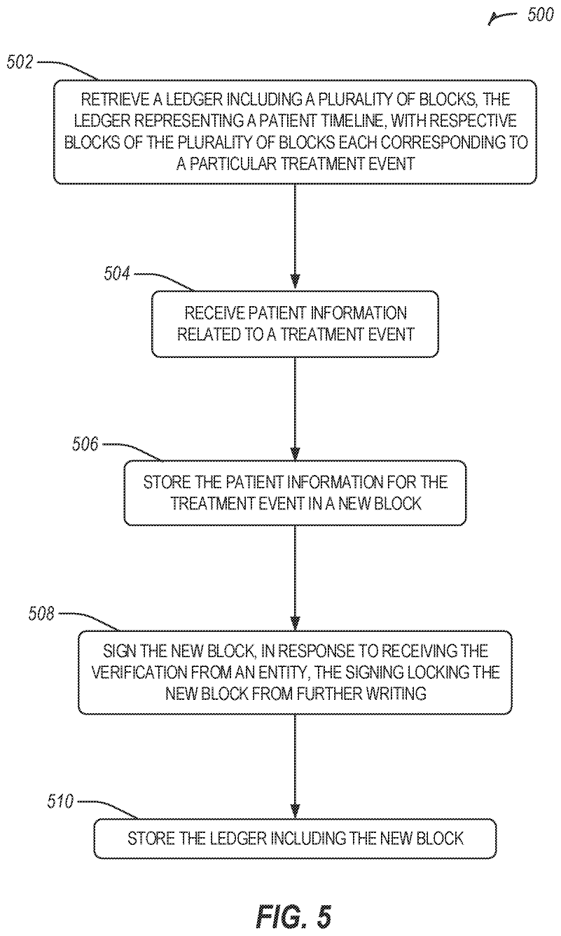

FIG. 5 illustrates a flowchart illustrating a technique 500 for tracking a patient throughout a treatment journey in accordance with some embodiments. The technique 500 includes an operation 502 to retrieve a ledger including a plurality of blocks, the ledger representing a patient timeline, with respective blocks of the plurality of blocks each corresponding to a particular treatment event. The ledger may be assigned to a single patient, for example for a single treatment journey (e.g., from initial treatment for an orthopedic procedure, such as a knee arthroplasty through physical therapy after a surgical intervention, or optionally to a revision procedure and therapy after the revision, or for life), or for more than one treatment journey (e.g., for a left knee and a right knee arthroplasty).

The technique 500 includes an operation 504 to receive patient information related to a treatment event. The patient information may include a diagnosis, symptom notes, medical imaging, a smart contract, a 3D model (e.g., of an implant), manufacturing data, metadata about a device (e.g., an implant), or the like.

The technique 500 includes an operation 506 to store the patient information for the treatment event in a new block. The patient information may include data from an event (e.g., surgery) or more granular data, such as a single medical image.

The technique 500 may include requesting verification of the new block, for example from an entity that is an authority on the patient information (e.g., in the new block), such as a surgeon, manufacturer, radiologist, 3D modeling generator (person or company), etc. In an example, the patient information stored in the new block may include a medical image and the entity may include a radiologist. In another example, the patient information stored in the new block may include a personalized implant and the entity may include a 3D model expert, a surgeon, or a manufacturer. In yet another example, the patient information stored in the new block may include an implant or guide, and the entity may include a manufacturer. In still another example, the patient information stored in the new block may include a diagnosis, and the entity may include a doctor (e.g., a surgeon). In another example, the patient information stored in the new block includes information related to a completed surgery and the entity may include a surgeon. In an example, the patient information stored in the new block includes patient history information and the entity includes a patient or someone entering the patient information (e.g., an assistant, a nurse, etc.).

The technique 500 includes an operation 508 to sign the new block, in response to receiving the verification from an entity, the signing locking the new block from further writing. The entity may include more than one person, company, etc. Signing the new block may include hashing the new block. In an example, after the new block is signed, the technique 500 may include revoking access by the entity to the ledger. For example, when a manufacturer has completed signing the new block after creation of a personal guide, access to the ledger may be revoked for the manufacturer, such that further patient information cannot be accessed. In some examples, access to other blocks in the ledger may be only in hashed or obscured signed form to protect patient privacy. Storing blocks as hashes allows retention of information (e.g., each entity having access to the ledger is able to verify that the blocks in the ledger have not changed or identify a change in a block based on the hash), but prevents exposure of patient data to entities that do not need access or limits access.

In an example, the new block may be appended to the ledger. The technique 500 includes an operation 510 to store the ledger including the new block. Storing the ledger may include saving the ledger, distributing an update (e.g., a notification with the new block) or an updated version of the ledger, publishing the updated ledger, or the like. The ledger may be voted on by other members of a distribution chain to ensure accuracy. The new block may be hashed before storing. In an example, keys and hashes for the new block or the plurality of blocks may be stored in a secure centralized database, such as in a hash table.

In case of an error in a signed block (e.g., the new block or an older block in the ledger), a new branch of the ledger may be created, for example with a corrected block. Creating the new branch may include maintaining the error block, such as for auditing purposes, for example with an indication that the error block includes and error and has been modified in the corrected block. In an example, creating a new branch may be an option that is limited only to the original creator or signer of the error block.

The technique 500 may include limiting access by the entity to blocks in the ledger that existed at the time the new block was signed or preventing access to any blocks (e.g., before or after the new block) after the new block is signed, added, or stored. In another example, the entity may be limited to accessing only the new block or only the new block and blocks related to the new block (e.g., for a same surgical procedure, same date, same treatment journey, etc.).

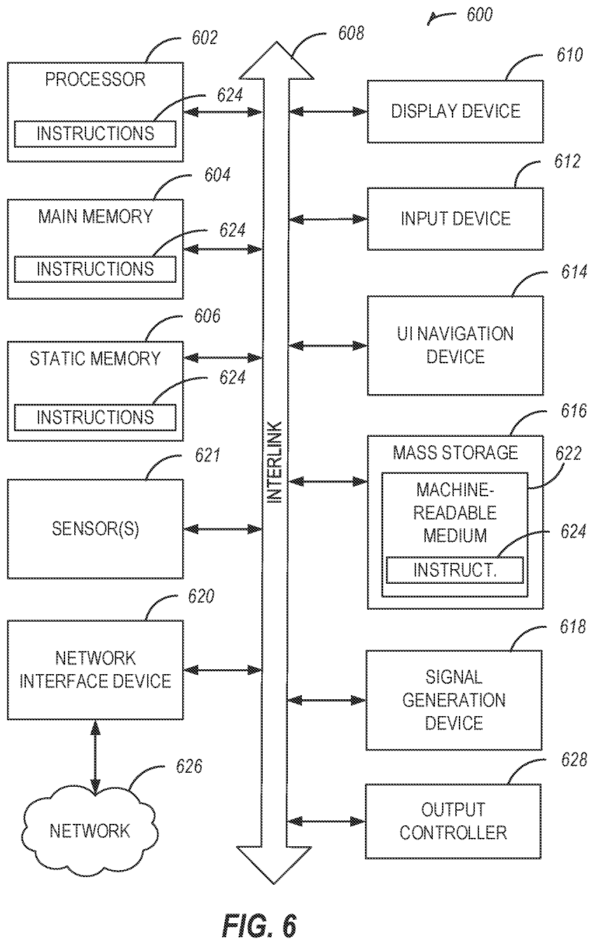

FIG. 6 illustrates a block diagram of an example machine 600 upon which any one or more of the techniques discussed herein may perform in accordance with some embodiments. In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624. The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine readable medium examples may include solid-state memories, and optical and magnetic media.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Each of these non-limiting examples may stand on its own, or may be combined in various permutations or combinations with one or more of the other examples.

Example 1 is a method for tracking a patient throughout a treatment journey, the method comprising: retrieving a ledger including a plurality of blocks, the ledger representing a patient timeline, with respective blocks of the plurality of blocks each corresponding to a particular treatment event; receiving patient information related to a treatment event; storing the patient information for the treatment event in a new block; requesting verification of the new block from an entity that is an authority on the patient information; signing the new block, in response to receiving the verification from the entity, the signing locking the new block from further writing; appending the new block to the ledger; and saving the ledger including the new block.

In Example 2, the subject matter of Example 1 includes, wherein the ledger is assigned to a single patient for a single treatment journey.

In Example 3, the subject matter of Examples 1-2 includes, wherein the patient information includes at least one of a diagnosis, symptom notes, medical imaging, a smart contract, a three-dimensional model (e.g., of an implant), manufacturing data, metadata about a device (e.g., an implant), or the like.

In Example 4, the subject matter of Examples 1-3 includes, wherein the patient information stored in the new block is a medical image and the entity is a radiologist.

In Example 5, the subject matter of Examples 1-4 includes, wherein the patient information stored in the new block is a personalized implant and the entity includes a three-dimensional model expert, a surgeon, and a manufacturer.

In Example 6, the subject matter of Examples 1-5 includes, wherein the patient information stored in the new block is an implant and the entity is a manufacturer.

In Example 7, the subject matter of Examples 1-6 includes, wherein the patient information stored in the new block is a diagnosis and the entity is a doctor.

In Example 8, the subject matter of Examples 1-7 includes, wherein the patient information stored in the new block includes information related to a completed surgery and the entity is a surgeon.

In Example 9, the subject matter of Examples 1-8 includes, wherein the patient information stored in the new block includes patient history information and the entity is a patient.

In Example 10, the subject matter of Examples 1-9 includes, wherein in case of an error in a signed block, a new branch of the ledger is created with a corrected block, while maintaining the error block.

In Example 11, the subject matter of Examples 1-10 includes, revoking access by the entity to the ledger after the new block is signed.

In Example 12, the subject matter of Examples 1-11 includes, limiting access by the entity to blocks in the ledger that existed at the time the new block was signed and preventing access from any blocks added after the new block.

In Example 13, the subject matter of Examples 1-12 includes, limiting access by the entity to blocks in the ledger related to the new block.

In Example 14, the subject matter of Examples 1-13 includes, wherein the new block is hashed before appending to the ledger.

In Example 15, the subject matter of Example 14 includes, wherein keys and hashes for the plurality of blocks are stored in a secure centralized database in a hash table.

Example 16 is a system configured to perform operations of any of any of the methods of Examples 1-15.

Example 17 is at least one machine-readable medium including instructions for operation of a computing system, which when executed by a machine, cause the machine to perform operations of any of the methods of Examples 1-15.

Example 18 is an apparatus comprising means for performing any of the methods of Examples 1-15.

Example 19 is a system for tracking a patient throughout a treatment journey, the system comprising: one or more processors coupled to a memory device, the memory device containing instructions that, when executed by the one or more processors, cause the system to: retrieve a ledger including a plurality of blocks, the ledger representing a patient timeline, with respective blocks of the plurality of blocks each corresponding to a particular treatment event; receive patient information related to a treatment event; store the patient information for the treatment event in a new block; request verification of the new block from an entity that is an authority on the patient information; sign the new block, in response to receiving the verification from the entity, the signing locking the new block from further writing; append the new block to the ledger; and save the ledger including the new block.

Example 20 is a non-transitory machine readable medium including instructions to, when executed by an electronic device, cause the electronic device to perform operations comprising: retrieve a ledger including a plurality of blocks, the ledger representing a patient timeline, with respective blocks of the plurality of blocks each corresponding to a particular treatment event; receive patient information related to a treatment event; store the patient information for the treatment event in a new block; request verification of the new block from an entity that is an authority on the patient information; sign the new block, in response to receiving the verification from the entity, the signing locking the new block from further writing; append the new block to the ledger; and save the ledger including the new block.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A method for tracking a patient throughout a treatment journey, the method comprising:
retrieving a ledger including a plurality of blocks, the ledger representing a patient timeline, with respective blocks of the plurality of blocks each corresponding to a particular treatment event;
receiving patient information related to a treatment event;
storing the patient information for the treatment event in a new block;
requesting verification of the new block from an entity that is an authority on the patient information;
signing the new block, in response to receiving the verification from the entity, the signing locking the new block from further writing;
appending the new block to the ledger; and
storing the ledger including the new block.

2. The method of claim 1, wherein the ledger is assigned to a single patient for a single treatment journey.

3. The method of claim 1, wherein the patient information includes at least one of a diagnosis, symptom notes, medical imaging, a smart contract, a 3D model of an implant, manufacturing data, or metadata about a device.

4. The method of claim 1, wherein the patient information stored in the new block is a medical image and the entity is a radiologist.

5. The method of claim 1, wherein the patient information stored in the new block is a personalized implant and the entity includes a 3D model expert, a surgeon, and a manufacturer.

6. The method of claim 1, wherein the patient information stored in the new block is an implant and the entity is a manufacturer.

7. The method of claim 1, wherein the patient information stored in the new block is a diagnosis and the entity is a doctor.

8. The method of claim 1, wherein the patient information stored in the new block includes information related to a completed surgery and the entity is a surgeon.

9. The method of claim 1, wherein the patient information stored in the new block includes patient history information and the entity is a patient.

10. The method of claim 1, wherein in case of an error in a signed block, a new branch of the ledger is created with a corrected block, while maintaining the error block.

11. The method of claim 1, further comprising revoking access by the entity to the ledger after the new block is signed.

12. The method of claim 1, further comprising limiting access by the entity to blocks in the ledger that existed at the time the new block was signed and preventing access from any blocks added after the new block.

13. The method of claim 1, further comprising limiting access by the entity to blocks in the ledger related to the new block.

14. The method of claim 1, wherein the new block is hashed before appending to the ledger.

15. The method of claim 14, wherein keys and hashes for the plurality of blocks are stored in a secure centralized database in a hash table.

16. A system for tracking a patient throughout a treatment journey, the system comprising:
one or more processors coupled to a memory device, the memory device containing instructions that, when executed by the one or more processors, cause the system to:
retrieve a ledger including a plurality of blocks, the ledger representing a patient timeline, with respective blocks of the plurality of blocks each corresponding to a particular treatment event;
receive patient information related to a treatment event;
store the patient information for the treatment event in a new block;
request verification of the new block from an entity that is an authority on the patient information;
sign the new block, in response to receiving the verification from the entity, the signing locking the new block from further writing;
append the new block to the ledger; and
store the ledger including the new block.

17. The system of claim 16, wherein the ledger is assigned to a single patient for a single treatment journey.

18. The system of claim 16, wherein the patient information includes at least one of a diagnosis, symptom notes, medical imaging, a smart contract, a 3D model of an implant, manufacturing data, or metadata about a device.

19. The system of claim 16, wherein the patient information stored in the new block is a medical image and the entity is a radiologist.

20. A non-transitory machine readable medium including instructions to, when executed by an electronic device, cause the electronic device to perform operations comprising:
retrieve a ledger including a plurality of blocks, the ledger representing a patient timeline, with respective blocks of the plurality of blocks each corresponding to a particular treatment event;
receive patient information related to a treatment event;
store the patient information for the treatment event in a new block;
request verification of the new block from an entity that is an authority on the patient information;
sign the new block, in response to receiving the verification from the entity, the signing locking the new block from further writing;
append the new block to the ledger; and
store the ledger including the new block.
